(19) United States
(12) Patent Application Publication
(10) Pub. No.: US 2009/0126126 A1
(43) Pub. Date: May 21, 2009

(54) COMPOSITION FOR THE OXIDATION DYEING OF KERATIN FIBERS COMPRISING AT LEAST ONE CELLULOSE WITH A LEAST ONE HYDROPHOBIC SUBSTITUENT, AT LEAST ONE OXIDATION DYE AND AT LEAST ONE CATIONIC POLYMER, AND METHODS OF USE THEREOF

(76) Inventors: Marie-Pascale AUDOUSSET, Asnieres (FR); Jean-Marc Ascione, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/267,744

(22) Filed: Nov. 10, 2008

(30) Foreign Application Priority Data
Nov. 9, 2007 (FR) 0758931

(51) Int. Cl. A61K 8/72; A61Q 5/10

(52) U.S. Cl. 8/407; 8/406; 8/421

(57) ABSTRACT

The present disclosure relates to a novel dye composition for keratin fibers, comprising, in a medium suitable for dyeing: (A) at least one nonionic derivative of cellulose comprising at least one hydrophobic substituent comprising from 8 to 30 carbon atoms; (B) at least one oxidation dye, and (C) at least one specific cationic polymer; and to a process for the oxidation dyeing of keratin fibers using said composition.

This application claims benefit of priority under 35 U.S.C. § 119 to French Patent Application No. FR 0 758 931, filed Nov. 9, 2007, the contents of which are incorporated herein by reference.

Disclosed herein is a composition for the oxidation dyeing of keratin fibers.

It is known practice to dye keratin fibers, including human hair, with dye compositions comprising oxidation dye precursors, generally known as oxidation bases, such as ortho- or para-phenylenediamines, ortho- or para-aminophenols and heterocyclic compounds. These oxidation bases are colorless or weakly colored compounds which, in combination with oxidizing products, can give rise, via an oxidative condensation process, to colored compounds.

It is also known that it is possible to vary the shades obtained with these oxidation bases by combining them with couplers or coloring modifiers, the latter being chosen, for example, from aromatic meta-diamines, meta-aminophenols, meta-diphenols, and certain heterocyclic compounds such as indole compounds.

The variety of the molecules involved as oxidation bases and couplers makes it possible to obtain a rich palette of colors.

The “permanent” coloring obtained by virtue of these oxidation dyes should, moreover, meet a certain number of requirements.

For example, it should have no toxicological drawbacks, it should allow shades to be obtained in the desired strength, and it should show good color-fastness with respect to external agents such as light, bad weather, washing, permanent-waving, perspiration, and rubbing.

The dyes should also allow white hair to be covered and, finally, should be as nonselective as possible, i.e. they should make it possible to obtain the smallest possible differences in coloring along the same keratin fiber, which is generally differently sensitized (i.e. damaged) between its tip and its root.

Moreover, it is desirable that the compositions obtained should, in addition, have good rheological properties, while at the same time conserving good coloring properties. For instance, these compositions should not run on the face or out of the areas intended to be dyed, when they are applied, such as after mixing with an oxidizing agent.

Improving the power of dyeing by combining a para-phenylene diamine oxidation base and at least one nonionic amphiphilic polymer such as hydroxycellulose modified with a hydrophobic group is discussed in International Patent Application No WO 98/03150.

However, these compositions do not entirely meet the above-mentioned requirements and can be improved, for example in terms of dyeing properties, further for example in terms of dyeing selectivity and power. Thus, there is a need in the art to obtain stable hair dyeing compositions, such as, in the form of creams, which are easy to prepare and to apply, which may comprise high concentrations of dyes in the form of salts, which have good rheological qualities, and which produce strong, relatively nonselective colorations that withstand the various attacks that keratin fibers may be subjected to.

Accordingly, one aspect of the present disclosure is a dye composition for keratin fibers, including human keratin fibers such as the hair, that meets at least one of the conditions discussed above, comprising, in a medium suitable for dyeing:

(A) at least one nonionic derivative of cellulose comprising at least one hydrophobic substituent comprising from 8 to 30 carbon atoms;

(B) at least one oxidation dye, and

(C) at least one cationic diquaternary ammonium polymer comprising repeat units of formula (I):

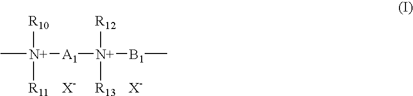

wherein:

R₁₀, R₁₁, R₁₂ and R₁₃, which may be identical or different, are chosen from aliphatic, alicyclic, and arylaliphatic C₁-C₂₀ radicals and hydroxyalkylaliphatic radicals wherein the alkyl radical is C₁-C₄, or alternatively, R₁₀, R₁₁, R₁₂ and R₁₃, together or separately, constitute, with the nitrogen atoms to which they are attached, heterocycles optionally comprising a second heteroatom other than nitrogen, or alternatively R₁₀, R₁₁, R₁₂ and R₁₃ are chosen from linear and branched C₁-C₆ alkyl radicals substituted with a nitrite, ester, acyl, amide, —CO—O—R₁₄-D or —CO—NH—R₁₄-D group, where R₁₄ is an alkylene and D is a quaternary ammonium group;

A₁ and B₁ are chosen from linear and branched, saturated and unsaturated C₂-C₂₀ polymethylene groups that may optionally comprise, linked to or intercalated in the main chain, at least one aromatic ring, and/or at least one entity chosen from oxygen and sulphur atom, and sulphoxide, sulphone, disulphide, amino, alkylamino, hydroxyl, quaternary ammonium, ureido, amide, and ester groups, and

X⁻¹ is an anion derived from a mineral or organic acid;

A₁, R₁₀ and R₁₂ can form, with the two nitrogen atoms to which they are attached, a piperazine ring;

wherein, if A₁ is chosen from linear and branched, saturated and unsaturated alkylene and hydroxyalkylene radicals, then B₁ may also be chosen from —(CH₂)ₙ—CO-D-OC—(CH₂)ₙ— groups wherein n ranges from 1 to 100, for instance ranging from 1 to 50, and D is chosen from:

a) glycol residues of formula: —O-Z-O— where Z is chosen from linear and branched hydrocarbon-based radicals and groups of the formulae: —(CH₂—CH₂—O)ₓ—CH₂—CH₂— and —[CH₂—CH(CH₃)—O]_{y}—CH₂—CH(CH₃)— where x and y denote an integer ranging from 1 to 4, representing a defined and unique degree of polymerization, or any number ranging from 1 to 4, representing a mean degree of polymerization;

b) bissecondary diamines residue such as a piperazine derivative;

c) bisprimary diamines residue of formula: —NH—Y—NH—, where Y is chosen from linear and branched hydrocarbon-based radicals, and the radical —CH₂—CH₂—S—S—CH₂—CH₂—; and

d) ureylene groups of formula: —NH—CO—NH—.

The dye compositions according to the present disclosure can have at least one of the following properties:

they make it possible to obtain compositions with a viscosity of a cream, which are stable over time,

they stand out by virtue of the fact that they could be easily mixed with the oxidizing composition,

they stand out by virtue of the rheological qualities of the creams obtained (good viscosity of cream as a mixture),

they are easy to apply after mixing with the oxidizing composition at the time the dyeing is carried out (qualities of use on the head).

In addition, the compositions according to the present disclosure may make it possible to obtain compositions capable of producing colorings with varied, chromatic, powerful, aesthetic and relatively nonselective shades which are uniform over all the keratin fibers, including human keratin fibers such as the hair, and which are highly resistant to the various attacks to which the fibers may be subjected.

Another aspect of the present disclosure comprises a process for the dyeing of keratin fibers, wherein the cosmetic composition according to the present disclosure is used.

Still another aspect of the present disclosure relates to the method of use of this cosmetic composition for dyeing keratin fibers, including human keratin fibers such as the hair.

Other features, aspects, subjects and benefits of the present disclosure will emerge more clearly on reading the description and the non-limiting examples which follow.

Unless otherwise indicated, the limits of the ranges of values which are given in the context of the present disclosure are included in these ranges.

As used herein, “derivative(s) of cellulose” is intended to mean at least one compound comprising at least one cellobiose unit having the structure:

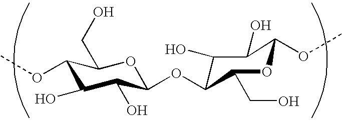

wherein at least one hydroxyl group may be substituted.

The at least one nonionic derivative of cellulose with at least one hydrophobic substituent in accordance with the present disclosure is chosen from amphiphilic polymers that are associative in nature. It may comprise hydrophilic units and hydrophobic units and are capable of interacting and of associating with other nonionic derivatives of cellulose or with other molecules, reversibly, by virtue of the presence of their hydrophobic chains.

For example, the at least one derivative of cellulose of the present disclosure can be a cellulose ether comprising at least one hydrophobic substituent comprising from 8 to 30 carbon atoms.

The at least one nonionic derivative of cellulose with at least one hydrophobic substituent in accordance with the present disclosure can be, for example, prepared from water-soluble nonionic ethers of cellulose, wherein all or some of the reactive hydroxyl functional groups are substituted with at least one hydrophobic chain comprising from 8 to 30 carbon atoms, for example from 10 to 22 carbon atoms, and further for example 16 carbon atoms. The reaction steps involved in the preparation of the at least one cellulose derivative of the present disclosure are known to those skilled in the art.

The nonionic ethers of cellulose chosen for preparing the at least one nonionic derivative of cellulose with at least one hydrophobic substituent according to the present disclosure can have, for instance, a degree of nonionic substitution, for example of at least one group chosen from methyl, hydroxyethyl and hydroxypropyl groups, that is sufficient to be water-soluble, i.e. to form a substantially clear solution when they are dissolved in water at 25° C. at the concentration of 1% by weight.

The nonionic ethers of cellulose chosen for preparing the at least one nonionic derivative of cellulose with at least one hydrophobic substituent according to the present disclosure can have, for example, a relatively low number-average molar mass, of less than 800,000 g/mol, such as ranging from 50,000 to 700,000 g/mol, and further for example ranging from 200,000 to 600,000 g/mol.

In at least one embodiment, the at least one cellulose derivative of the present disclosure is a hydroxyethylcellulose comprising at least one hydrophobic substituent comprising from 8 to 30 carbon atoms.

The at least one nonionic derivative of cellulose used according to the present disclosure are substituted with at least one group chosen from aliphatic and aromatic, saturated and unsaturated, linear, branched and cyclic C₈-C₃₀ hydrocarbon groups, that may be attached to the cellulose ether substrate via an ether, ester or urethane bond, and in at least one embodiment an ether bond.

According to one embodiment, the at least one hydrophobic substituent used as a substituent of the at least one nonionic derivative of cellulose according to the present disclosure is C₈-C₃₀, such as C₁₀-C₂₂, alkyl, arylalkyl and alkylaryl groups.

For example, the at least one hydrophobic substituent according to the present disclosure can be chosen from saturated alkyl chains.

According to a at least one embodiment, the at least one hydrophobic substituent according to the present disclosure is a cetyl group.

The at least one nonionic derivative of cellulose with at least one hydrophobic substituent according to the present disclosure may, for example, have a viscosity ranging from 100 to 100,000 mPa·s, and further for example from 200 to 20,000 mPa·s, measured at 25° C. in a solution at 1% by weight of polymer in water, this viscosity being determined conventionally using a Brookfield LVT viscometer at 6 rpm with the No. 3 spindle.

The degree of hydrophobic substitution of the at least one hydrophilic nonionic derivative of cellulose used according to the present disclosure may, for example, range from 0.1% to 10% by weight, such as from 0.1% to 1% by weight, and further for example from 0.4% to 0.8% by weight, of the total weight of the polymer.

Non-limiting examples of the nonionic derivatives of cellulose with at least one hydrophobic substituent that can be used in the compositions of the present disclosure include the cetyl hydroxyethylcelluloses sold under the names NATROSOL® Plus Grade 330 CS and POLYSURF® 67 CS (INCI: Cetyl Hydroxyethylcellulose) by the company Aqualon/Hercules.

The at least one nonionic derivative of cellulose with at least one hydrophobic substituent of the compositions according to the present disclosure may, for example, be present in an amount ranging from 0.01% to 10% by weight, further for example from 0.05% to 3% by weight, and such as from 0.1% to 1% by weight, relative to the total weight of the composition.

The at least one oxidation dye that can be used in the present disclosure may be chosen from oxidation bases and couplers.

The at least one oxidation dye that can be used according to the present disclosure can be, for example, chosen from oxidation bases, oxidation couplers, and addition salts thereof.

By way of non-limiting example, the at least one oxidation base can be chosen from para-phenylenediamines, bisphenylalkylenediamines, para-aminophenols, bis-para-aminophenols, ortho-aminophenols, heterocyclic bases, and addition salts thereof.

Among the para-phenylenediamines, mention may be made, by way of non-limiting example, of para-phenylenediamine, para-toluoylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylene-diamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(2′-hydroxypropyl)-para-phenylenediamine, 2-hydroxymethyl-para-phenylenediamine, N,N-dimethyl-3-methyl-para-phenylenediamine, N,N-(ethyl-2′-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(2′-hydroxyethyloxy)-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and the acid addition salts thereof.

In at least one embodiment, at least one oxidation dye is a para-phenylenediamine chosen from para-phenylenediamine, para-toluoylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and the acid addition salts thereof with an acid.

Among the bisphenylalkylenediamines, mention may, by way of non-limiting example, be made of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-amino-phenyl)tetramethylenediamine, N,N′-bis-(4-methylaminophenyl)tetramethylenediamine, N,N′-bis(ethyl)-N,N′-bis(4′-amino-3′-methylphenyl)ethylenediamine, 1,8-bis(2,5-diamino-phenoxy)-3,6-dioxaoctane, and the acid addition salts thereof.

Among the para-aminophenols, mention may be made, by way of non-limiting example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxy-ethylaminomethyl)phenol, 4-amino-2-fluorophenol, and the acid addition salts thereof.

Among the ortho-aminophenols, mention may, by way of non-limiting example, be made of 2-aminophenol, 2-amino-5-methylphenol, 2-amino-6-methylphenol, 5-acetamido-2-aminophenol, and the acid addition salts thereof.

Among the heterocyclic bases, mention may, by way of non-limiting example, be made of pyridine derivatives, pyrimidine derivatives, pyrazole derivatives, pyrazolone derivatives, and addition salts thereof.

Among the pyridine derivatives, non-limiting mention may be made of the compounds described, for example, in British Patent Nos. GB 1 026 978 and 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diamino-pyridine, and acid addition salts thereof.

Other pyridine oxidation bases that can be used in the present disclosure are the 3-aminopyrazolo[1,5-a]pyridine oxidation bases and addition salts thereof described, for example, in French Patent Application FR 2 801 308. By way of non-limiting example, mention may be made of pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-aminopyrazolo[1,5-a]pyridin-2-carboxylic acid; 2-methoxypyrazolo[1,5-a]pyridin-3-ylamino; (3-amino-pyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl)(2-hydroxyethyl)amino]ethanol; 2-[(3-aminopyrazolo[1,5-a]pyridin-7-yl)(2-hydroxyethyl)amino]ethanol; 3-aminopyrazolo[1,5-a]pyridin-5-ol; 3-amino-pyrazolo[1,5-a]pyridin-4-ol; 3-aminopyrazolo[1,5-a]pyridin-6-ol; 3-aminopyrazolo[1,5-a]pyridin-7-ol; and also addition salts thereof with an acid or with a base.

Among the pyrimidine derivatives, non-limiting mention may be made of the compounds described, for example, in German Patent No. DE 2359399; Japanese Patent No. JP 88-169571; Japanese Patent No. JP 05-63124; European Patent No. EP 0770375 or International Patent Application No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and pyrazolopyrimidine derivatives such as those mentioned in French Patent Application No. FR-A-2 750 048 and among which non-limiting mention may be made of pyrazolo-[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethyl-pyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine, and addition salts thereof with an acid and tautomeric forms thereof when a tautomeric equilibrium exists.

Among the pyrazole derivatives that can be used, non-limiting mention may be made, for example, of the compounds described in German Patent No. DE-A-38 43 892, German Patent No. DE-A-41 33 957, International Patent Application No. WO 94/08969, International Patent Application No. WO 94/08970, French Patent Application No. FR-A-2 733 749 and German Patent Application No. DE-A-1 95 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethyl-pyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-amino-ethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and addition salts thereof.

Among the pyrazolone derivatives that can be used, non-limiting mention may, for example, be made of the following compounds and addition salts thereof:
2,3-diaminodihydropyrazolone;
4,5-diamino-1,2-dimethyl-1,2-dihydropyrazol-3-one;
4-amino-5-methylamino-1,2-dimethyl-1,2-dihydropyrazol-3-one;
4-amino-5-dimethylamino-1,2-dimethyl-1,2-dihydropyrazol-3-one;
4-amino-5-(2-hydroxyethyl)amino-1,2-dimethyl-1,2-dihydropyrazol-3-one;
4-amino-5-(pyrrolidin-1-yl)-1,2-dimethyl-1,2-dihydropyrazol-3-one;
4-amino-5-(piperidin-1-yl)-1,2-dimethyl-1,2-dihydropyrazol-3-one;
4,5-diamino-1,2-di(2-hydroxyethyl)-1,2-dihydropyrazol-3-one;
4-amino-5-methylamino-1,2-di(2-hydroxyethyl)-1,2-dihydropyrazol-3-one;
4-amino-5-dimethylamino-1,2-di(2-hydroxyethyl)-1,2-dihydropyrazol-3-one;
4-amino-5-(2-hydroxyethyl)amino-1,2-di(2-hydroxyethyl)-1,2-dihydropyrazol-3-one;
4-amino-5-(pyrrolidin-1-yl)-1,2-di(2-hydroxyethyl)-1,2-dihydropyrazol-3-one;
4-amino-5-(piperidin-1-yl)-1,2-di(2-hydroxyethyl)-1,2-dihydropyrazol-3-one;
4,5-diamino-1,2-diethyl-1,2-dihydropyrazol-3-one;
4,5-diamino-1,2-diphenyl-1,2-dihydropyrazol-3-one;
4,5-diamino-1-ethyl-2-methyl-1,2-dihydropyrazol-3-one;
4,5-diamino-2-ethyl-1-methyl-1,2-dihydropyrazol-3-one;
4,5-diamino-1-phenyl-2-methyl-1,2-dihydropyrazol-3-one;
4,5-diamino-2-phenyl-1-methyl-1,2-dihydropyrazol-3-one;
4,5-diamino-1-(2-hydroxyethyl)-2-methyl-1,2-dihydropyrazol-3-one;
4,5-diamino-2-(2-hydroxyethyl)-1-methyl-1,2-dihydropyrazol-3-one;
2,3-diamino-6,7-dihydro-1H,5H-pyrazolo[1,2-a]pyrazol-1-one;
2-amino-3-methylamino-6,7-dihydro-1H,5H-pyrazolo[1,2-a]pyrazol-1-one;
2-amino-3-dimethylamino-6,7-dihydro-1H,5H-pyrazolo[1,2-a]pyrazol-1-one;
2-amino-3-ethylamino-6,7-dihydro-1H,5H-pyrazolo[1,2-a]pyrazol-1-one;
2-amino-3-isopropylamino-6,7-dihydro-1H,5H-pyrazolo[1,2-a]pyrazol-1-one;
2-amino-3-(2-hydroxyethyl)amino-6,7-dihydro-1H,5H-pyrazolo[1,2-a]pyrazol-1-one;
2-amino-3-(2-hydroxypropyl)amino-6,7-dihydro-1H,5H-pyrazolo[1,2-a]pyrazol-1-one;
2-amino-3-bis(2-hydroxyethyl)amino-6,7-dihydro-1H,5H-pyrazolo[1,2-a]pyrazol-1-one;
2-amino-3-(pyrrolidin-1-yl)-6,7-dihydro-1H,5H-pyrazolo[1,2-a]pyrazol-1-one;
2-amino-3-(3-hydroxypyrrolidin-1-yl)-6,7-dihydro-1H,5H-pyrazolo[1,2-a]pyrazol-1-one;
2-amino-3-(piperidin-1-yl)-6,7-dihydro-1H,5H-pyrazolo[1,2-a]pyrazol-1-one;
2,3-diamino-6-hydroxy-6,7-dihydro-1H,5H-pyrazolo[1,2-a]pyrazol-1-one;
2,3-diamino-6-methyl-6,7-dihydro-1H,5H-pyrazolo[1,2-a]pyrazol-1-one;
2,3-diamino-6,6-dimethyl-6,7-dihydro-1H,5H-pyrazolo[1,2-a]pyrazol-1-one;
2,3-diamino-5,6,7,8-tetrahydro-1H,6H-pyridazino[1,2-a]pyrazol-1-one;
2,3-diamino-5,8-dihydro-1H,6H-pyridazino[1,2-a]pyrazol-1-one;
4-amino-5-dimethylamino-1,2-diethyl-1,2-dihydropyrazol-3-one;
4-amino-1,2-diethyl-5-ethylamino-1,2-dihydropyrazol-3-one;
4-amino-1,2-diethyl-5-isopropylamino-1,2-dihydropyrazol-3-one;
4-amino-1,2-diethyl-5-(2-hydroxyethylamino)-1,2-dihydropyrazol-3-one;
4-amino-5-(2-dimethylaminoethylamino)-1,2-diethyl-1,2-dihydropyrazol-3-one;
4-amino-5-[bis(2-hydroxyethyl)amino]-1,2-diethyl-1,2-dihydropyrazol-3-one;
4-amino-1,2-diethyl-5-(3-imidazol-1-yl-propylamino)-1,2-dihydropyrazol-3-one;
4-amino-1,2-diethyl-5-(3-hydroxypyrrolidin-1-yl)-1,2-dihydropyrazol-3-one;
4-amino-5-pyrrolidin-1-yl-1,2-diethyl-1,2-dihydropyrazol-3-one;
4-amino-5-(3-dimethylaminopyrrolidin-1-yl)-1,2-diethyl-1,2-dihydropyrazol-3-one; and
4-amino-1,2-diethyl-5-(4-methylpiperazin-1-yl)pyrazolidin-3-one.

The at least one oxidation base can be present in an amount ranging from 0.001% to 20% by weight, for example from 0.005% to 10% by weight, and further for example from 0.01% to 5% by weight, relative to the total weight of the composition.

The at least one oxidation coupler present in the compositions of the present disclosure may be chosen from benzene couplers, heterocyclic couplers, naphthalene couplers, and addition salts thereof.

By way of non-limiting example of benzene couplers that can be used in the compositions according to the present disclosure, mention may be made of meta-aminophenols, meta-phenylenediamines, meta-diphenols, and also addition salts thereof.

Further among the couplers that may be used in the compositions according to the present disclosure, non-limiting mention may be made of 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, β-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N—(β-hydroxyethyl)amino-3,4-methylenedioxybenzene, 2,6-bis(β-hydroxyethylamino)toluene, and the acid addition salts thereof.

In at least one embodiment, the composition of the present disclosure comprises at least one oxidation base and at least one coupler.

The at least one oxidation coupler can be present in an amount ranging from 0.001 to 20% by weight, for example from 0.005% to 10% by weight, and further for example from 0.01% to 5% by weight, relative to the total weight of the composition.

The addition salts of the oxidation bases and of the couplers that can be used in the context of the present disclosure may, by way of non-limiting example, be chosen from the addition salts with an acid, such as the hydrochlorides, hydrobromides, sulphates, citrates, succinates, tartrates, lactates, tosylates, benzenesulphonates, phosphates, and acetates, and the addition salts with a base, such as sodium hydroxide, potassium hydroxide, aqueous ammonia, amines, and alkanolamines.

The composition comprises, in addition, at least one cationic polymer.

As used herein, “cationic polymer” is any polymer comprising cationic groups and/or groups that can be ionized to cationic groups.

The cationic charge density of the cationic polymers according to the disclosure can be, for example, greater than 1 meq/g. This charge density is determined by the Kjeldahl method. It can also be calculated from the chemical nature of the polymer.

In formula (I) as disclosed herein, X⁻ is an anion such as chloride or bromide.

The cationic polymers have a number-average molecular weight ranging from 1,000 to 100,000

Polymers of this type are, for instance, described in French Patent Nos. FR 2 320 330, FR 2 270 846, FR 2 316 271, FR 2 336 434, and FR 2 413 907, and U.S. Pat. No. 2,273,780, U.S. Pat. No. 2,375,853, U.S. Pat. No. 2,388,614, U.S. Pat. No. 2,454,547, U.S. Pat. No. 3,206,462, U.S. Pat. No. 2,261,002, U.S. Pat. No. 2,271,378, U.S. Pat. No. 3,874,870, U.S. Pat. No. 4,001,432, U.S. Pat. No. 3,929,990, U.S. Pat. No. 3,966,904, U.S. Pat. No. 4,005,193, U.S. Pat. No. 4,025,617, U.S. Pat. No. 4,025,627, U.S. Pat. No. 4,025,653, U.S. Pat. No. 4,026,945, and U.S. Pat. No. 4,027,020.

Non-limiting examples of the at least one cationic polymer include polymers which are constituted of repeat units of formula (II) below:

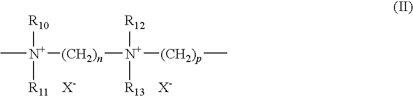

wherein R₁₀, R₁₁, R₁₂ and R₁₃, which may be identical or different, are chosen from C₁-C₄ alkyl and hydroxyalkyl radicals, n and p are integers ranging from 2 to 20, and X⁻ is an anion derived from a mineral or organic acid.

In at least one embodiment of the present disclosure, use is made of the polymers with repeat units of formula (W) and (U) below:

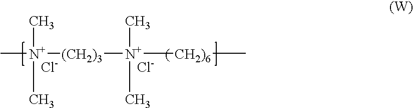

for example those of which the molecular weight, determined by gel permeation chromatography, ranges from 9,500 to 9,900; and

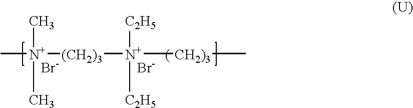

for example those of which the molecular weight, determined by gel permeation chromatography, is 1,200.

The at least one cationic polymer in the composition according to the present disclosure can be present in a total amount ranging from 0.01% to 10% by weight, relative to the weight of the composition, for example from 0.05% to 6%, further for example from 0.1% to 5% by weight, relative to the weight of the composition.

The composition of the present disclosure may comprise at least one fatty acid amide. The at least one fatty acid amide may, for example, be chosen from the amides of a C₂-C₁₀ alkanolamine and of a C₁₄-C₃₀ fatty acid, and further for example from the amides of a C₂-C₆ alkanolamine and of a C₁₄-C₂₂ fatty acid.

The at least one fatty acid amide is generally nonionic, i.e. it does not comprise any ionic charges.

The amide of an alkanolamine and of a C₁₄-C₃₀ fatty acid may, for example, be chosen from:

oleic acid diethanolamide, such as the amide sold under the trade name MEXANYL® GT by the company Chimex,

myristic acid monoethanolamide, such as the amide sold under the trade name COMPERLAN® MM by the company Cognis,

soybean fatty acid diethanolamide, such as the amide sold under the trade name COMPERLAN® VOD by the company Cognis,

stearic acid ethanolamide, such as the amide sold under the trade name MONAMID® S by the company Uniqema,

oleic acid monoisopropanolamide, such as the amide sold under the trade name WITCAMIDE® 61 by the company Witco,

linoleic acid diethanolamide, such as the amide sold under the trade name PURTON® SFD by the company Zschimmer & Schwarz,

stearic acid monoethanolamide, such as the amide sold under the trade name MONAMID® 972 by the company ICI/Uniqema,

behenic acid monoethanolamide, such as the amide sold under the trade name INCROMIDE® BEM by Croda,

isostearic acid monoisopropanolamide, such as the amide sold under the trade name WITCAMIDE® SPA by the company Witco,

erucic acid diethanolamide, such as the amide sold under the trade name erucic acid diethanolamide by the company Stéarineries Dubois,

ricinoleic acid monoethanolamide, such as the amide sold under the trade name ricinoleic monoethanolamide by the company Stéarineries Dubois.

The at least one fatty acid amide of the compositions according to the present disclosure may, for example, be present in an amount ranging from 0 to 10%, further for example from 0.2% to 10% by weight, and further for example from 0.5% to 6% by weight, relative to the total weight of the composition.

The dye composition in accordance with the present disclosure may also comprise at least one direct dye that may for example be chosen from nitrobenzene dyes, azo direct dyes, methine direct dyes, anthraquinone dyes, xanthene dyes, triarylmethane dyes, and addition salts thereof. These direct dyes may be nonionic, anionic or cationic in nature.

The medium used in the compositions according to the present disclosure can be an aqueous medium, or a medium comprising water and at least one organic solvent.

The at least one organic solvent used in the compositions according to the present disclosure may be chosen from monohydroxylated alcohols and polyols.

By way of monohydroxylated alcohols that can be used, non-limiting mention may be made of C₁-C₄ lower alcohols such as ethanol, isopropanol, tert-butanol, n-butanol, and mixtures thereof. In at least one embodiment, the alcohol used is ethanol.

By way of polyols that can be used, non-limiting mention may be made of propylene glycol, polyethylene glycols, and glycerol. By way of organic solvents, non-limiting mention may also be made of polyol ethers such as 2-butoxyethanol, propylene glycol monomethyl ether, diethylene glycol monoethyl ether, and diethylene glycol monomethyl ether, and also aromatic alcohols such as benzyl alcohol or phenoxyethanol, and mixtures thereof.

The at least one organic solvent in the compositions according to the present disclosure may be present in an amount ranging from 0 to 30%, for example from 0 to 20% by weight, relative to the total weight of the composition.

The compositions according to the present disclosure may also comprise at least one additional thickener, also referred to as a “rheology-adjusting agent”, different from the at least one nonionic derivative of cellulose with at least one hydrophobic substituent of the present disclosure.

The at least one rheology-adjusting agent may be chosen from mineral and organic thickeners, including polymeric associative thickeners, fatty alcohols different from those of the present disclosure, such as oleyl alcohol, cellulosic derivatives other than the at least one nonionic derivative of cellulose with at least one hydrophobic substituent according to the present disclosure (hydroxyethylcellulose, hydroxypropylcellulose, carboxymethylcellulose) and gums of microbial origin (such as xanthan gum, scleroglucan gum).

The at least one additional thickener can be present in an amount ranging from 0.01% to 20% by weight, for example from 1% to 10% by weight, relative to the total weight of the composition.

The dye composition in accordance with the present disclosure may also comprise at least one adjuvant conventionally used in compositions for dyeing the hair.

As used herein, “adjuvant” is intended to mean an additive different from the abovementioned compounds, such as anionic, cationic, nonionic, amphoteric, or zwitterionic surfactants or mixtures thereof; nonionic, amphoteric, zwitterionic, or anionic polymers, other than the at least one nonionic derivative of cellulose with at least one hydrophobic substituent according to the present disclosure, or mixtures of said polymers; penetrating agents; sequestering agents; fragrances; buffers; dispersants; conditioning agents such as, modified or unmodified, volatile or non-volatile silicones; film-forming agents; ceramides; preservatives; opacifiers; vitamins; amino acids; oligopeptides; peptides; modified or unmodified, hydrolysed or nonhydrolysed proteins; enzymes; branched or unbranched fatty acids and alcohols; animal, plant, or mineral waxes; hydroxylated organic acids; UV screens; antioxidants and free-radical scavengers; antidandruff agents; seborrhoea-regulating agents, calmatives; mineral, plant or animal oils; polyisobutenes and poly(α-olefins); pigments; acids, bases, plasticizers, mineral fillers, pearlescent agents, flakes; antistatic agents, and reducing agents.

The at least one adjuvant, may, for example, be present in an amount ranging from 0.01% to 40% by weight, further for example ranging from 0.1% to 25% by weight, relative to the total weight of the composition.

Those skilled in the art will take care to select this (or these) possible additional compound(s) in such a way that the beneficial properties intrinsically associated with the oxidation dyeing composition in accordance with the present disclosure are not, or not substantially, impaired by the addition(s) envisaged.

The pH of the dye composition in accordance with the present disclosure generally ranges from 3 to 12, and further for example from 5 to 11. It may be adjusted to the desired value via at least one acidifying agent or basifying agent commonly used in the dyeing of keratin fibers or by using at least one conventional buffer system.

Among the acidifying agents, mention may be made, by way of non-limiting example, of mineral or organic acids such as hydrochloric acid, orthophosphoric acid, sulphuric acid, sulphonic acids, and carboxylic acids, for instance acetic acid, tartaric acid, citric acid, and lactic acid.

Among the basifying agents, mention may, by way of non-limiting example, be made of aqueous ammonia, alkali metal carbonates, alkanolamines such as mono-, di-, and triethanolamines and derivatives thereof, sodium hydroxide or potassium hydroxide, and the compounds having the formula below:

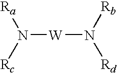

wherein:

W is a propylene residue optionally substituted with a hydroxyl group or a C₁-C₄ alkyl group;

Rₐ, R_{b}, R_{c}, and R_{d}, which may be identical or different, are chosen from hydrogen atoms, C₁-C₄ alkyl groups, and C₁-C₄ hydroxyalkyl groups.

The dye composition according to the present disclosure may be in various forms, such as in the form of creams or gels, or in any other form suitable for dyeing keratin fibers, including human hair.

The process for dyeing keratin fibers, of the present disclosure, is a process wherein the composition according to the present disclosure as defined above is applied to the fibers, for example in the presence of at least one oxidizing agent, for a period of time sufficient to develop the desired color. The color may be revealed at acidic, neutral, or alkaline pH and the at least one oxidizing agent may be added to the composition of the present disclosure just at the time of use, or it may be used starting from an oxidizing composition comprising it, applied simultaneously with or sequentially to the composition of the present disclosure.

According to at least one embodiment, the composition according to the present disclosure is a ready-to-use composition which is the dyeing composition mixed, for example at the time of use, with a composition comprising, in a medium suitable for dyeing, at least one oxidizing agent, the at least one oxidizing agent being present in a sufficient amount to develop a coloration. The mixture obtained is subsequently applied to the keratin fibers. After a leave-on time ranging from 3 to 50 minutes, for example from 5 to 30 minutes, the keratin fibers are rinsed, washed with shampoo, rinsed again, and then dried.

Non limiting examples of the at least one oxidizing agent conventionally used for the oxidation dyeing of keratin fibers include, for example, hydrogen peroxide, urea peroxide, alkali metal bromates, persalts such as perborates and persulphates, peracids and oxidase enzymes, among which non-limiting mention may be made of peroxidases, 2-electron oxidoreductases, such as uricases, and 4-electron oxygenases, such as laccases, these oxidoreductases being optionally combined with their customary cofactors, such as uric acid for uricases. In at least one embodiment, the oxidizing agent is hydrogen peroxide.

The oxidizing composition may also comprise at least one adjuvant conventionally used in compositions for dyeing the hair, as defined above.

The pH of the oxidizing composition comprising the at least one oxidizing agent is such that, after mixing with the dye composition, the pH of the resulting composition applied to the keratin fibers can, for example, range from 3 to 12, and further for example from 5 to 10. It may be adjusted to the desired value via at least one acidifying agent or basifying agent normally used in the dyeing of keratin fibers, as defined above.

The ready-to-use composition which is finally applied to the keratin fibers may be in other forms, such as in the form of creams or gels, or in any other form suitable for dyeing keratin fibers, for instance human keratin fibers such as the hair.

Another aspect of the present disclosure is a multicompartment dyeing device or dyeing “kit”, comprising at least one first compartment comprising at least one dye composition as defined above, and at least one second compartment comprising at least one oxidizing composition. This device may be equipped with a mechanism for delivering the desired mixture to the hair, such as the devices described in French Patent Application No. FR A 2 586 913.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples which follow serve to illustrate the present disclosure without, however, being limiting in nature.

### EXAMPLES

### Composition 1

The following compositions were prepared; the amounts indicated are in grams unless otherwise indicated.

| | |
|---|---|
| Resorcinol | 0.685 |
| Stearic acid monoethanolamide | 4.8 |
| Oleic acid | 3 |
| Cetyl hydroxyethylcellulose (NATROSOL ® plus grade | 0.4 |
| 330CS sold by Hercules) | |
| Aqueous ammonia containing 20% of NH₃ | 5 |
| Titanium dioxide | 0.3 |
| Ethanolamine | 0.8 |
| Oleth-10 | 1.8 |
| Aqueous solution containing 40% by weight of | 1.6 |
| polyquaternium-6 (polydimethyldiallylammonium | |
| chloride, MERQUAT ® 100 sold by Ondeo/Nalco) | |
| m-Aminophenol | 0.14 |
| EDTA | 0.2 |
| 2,4-Diaminophenoxyethanol HCl | 0.02 |
| Aqueous solution containing 60% by weight of | 1.2 |
| hexadimethrine chloride (MEXOMERE PO sold by | |
| the company Chimex) | |
| Reducing agent | qs |
| Antioxidant | qs |
| Hydroxypropylmethylcellulose | 0.191 |
| Oleth-30 | 1.5 |
| Steareth-2 | 5.5 |
| C₂₀-C₂₂ alcohols (NAFOL ® 2022 EN sold by | 3 |
| the company Sasol) | |
| Toluene-2,5-diamine | 0.7623 |
| Water | qs |

### Application Protocol

Each composition was mixed, extemporaneously, with one and a half times its weight of an oxidizing composition having a pH in the region of 3 (aqueous hydrogen peroxide at 20 volumes) (6% by weight of H₂O₂). The mixture was easily prepared and had a good viscosity; it was easily applied to grey hair, comprising 90% white hairs, at a rate of 10 g per 1 g of hair, and left on for 30 minutes. The hair was then rinsed, washed with a standard shampoo, and dried.

The hair coloration was evaluated visually. A hair coloration in a chestnut shade with a mahogany red tint was thus obtained.

These colorations had good properties, for instance in terms of selectivity and strength. The compositions obtained were stable over time.

### Composition 2

The following compositions were prepared; the amounts given are in grams unless otherwise indicated.

| | |
|---|---|
| Stearic acid monoethanolamide | 4.8 |
| Oleic acid | 3 |
| Cetyl hydroxyethylcellulose such as POLYSURF ® 67, | 0.5 |
| sold by Hercules | |
| p-Aminophenol | 0.24 |
| Aqueous ammonia containing 20% of NH₃ | 5 |
| Titanium dioxide | 0.3 |
| Ethanolamine | 0.8 |
| 2-Amino-3-hydroxypyridine | 0.2 |
| 6-Hydroxyindole | 0.01 |
| Aqueous solution containing 40% by weight of | 1.6 |
| dolydimethyldiallylammonium chloride (MERQUAT ® 100 | |
| sold by Ondeo/Nalco) | |
| 4-Amino-2-hydroxytoluene | 0.24 |
| 2-Methyl-5-hydroxyethylaminophenol | 0.15 |
| m-Aminophenol | 0.024 |
| EDTA | 0.2 |
| 2,4-Diaminophenoxyethanol HCl | 0.02 |
| Aqueous solution containing 60% by weight of | 1.2 |
| hexadimethrine chloride (MEXOMERE PO sold by | |
| the company Chimex) | |
| Reducing agent | qs |
| Antioxidant | qs |
| Hydroxypropylmethylcellulose | 0.191 |
| Oleth-30 | 1.5 |
| PEG-40 stearate | 1.8 |
| Steareth-2 | 5.5 |
| Glyceryl lauryl ether | 0.5 |
| C₂₀-C₂₂ alcohols (NAFOL ® 2022 EN sold by | 3 |
| the company Sasol) | |
| Toluene-2,5-diamine | 0.2772 |
| Water | qs |

The application to the hair was carried out according to the protocol of Example 1 above. A hair coloration in a blond shade with a coppery mahogany tint was thus obtained.

The Following Compositions were Prepared (Quantities Expressed in g % of Active Material)

| | Composition A | |
|---|---|---|
| | (present | |
| | disclosure) | Composition B |
| Ammonium Hydroxide | 4.115 | 4.115 |
| Erythorbic Acid | 0.5 | 0.5 |
| Ethanolamine | 0.7 | 0.7 |
| Edta | 0.2 | 0.2 |
| Sodium Sulfite | 0.5 | 0.5 |
| Titanium Dioxyde | 0.3 | 0.3 |
| 4-Amino-2-Hydroxytoluene | 0.246 | 0.246 |
| P-Phenylenediamine | 0.216 | 0.216 |
| Hexadimethrine Chloride | 1.2 MA | 1.2 MA |
| Cetyl Hydroxyethyicellulose | 0.45 | |
| Polyurethane-16 (Pur 6) | | 0.45 |
| Oleic Acid | 3 | 3 |
| Steareth-2 | 5.5 | 5.5 |
| Stearamide Mea (96) (And) | 5 | 5 |
| Ethanolamine (2) (And) | | |
| Stearic Acid (2) | | |
| Oleth-30 | 1.5 | 1.5 |
| Water | Qs 100 | Qs 100 |

At the time of use, each of the above compositions was mixed, weight for weight, with a 20 volume hydrogen peroxide solution.

Each of the resulting mixtures was then applied onto locks of natural hair with 90% of white hair (BN) or sensitized hair with 90% of white hair exhibiting an alkaline solubility of 22.9% (SA22.9), 15 g of composition for 1 g of hair. After 30 minutes, the hair was then rinsed with water, washed with a standard shampoo, rinsed again, and dried.

### Viscosity of the Compositions

The viscosity measurement was made with a Rheometer METTLER RM180 Rhéomat. The measures were made at 25° C., with a rotating speed of 200 rpm with a mobile
mobile M4 for the cream
mobile M3 or M2 after mixing with the oxidant with 20 volumes (6% H2O2) with a 1 to 1.5 ratio (measurement after 2 min mixing).

| | A | B |
|---|---|---|
| Viscosity of the cream M4 (Cps) | 6600 | 4700 |
| Viscosity of the mixture with | 500 | 160 |
| the oxidant M3 or M2 (Cps) | | |

The Composition B was too fluid when in the cream form and after mixing. Composition A was acceptable in cream form and fluid after mixing with the oxidant, facilitating the application of the mixture on the hair.

### Colorimetric Evaluation

The color of the hair was determined by using the L*a*b* system, with a MINOLTA CM2002® spectrophotometer.

According to this system, L* indicates the lightness. The lower the value of L*, the more intense is the color of the hair. The chromaticity coordinates are expressed by the parameters a* and b*, a* indicating the axis of red/green shades and b* the axis of yellow/blue shades.

### Selectivity of the Coloration

The selectivity of the coloration is the variation of the color between natural colored hair and permed colored hair. Natural hair is representative of the nature of the hair at the root, and the permed hair is representative of the nature of the hair at the tip.

The selectivity is measured by:

ΔE, which is the color variation between a natural colored lock and a permed colored lock, is obtained from the following formula:

ΔE=\sqrt{(L*−Lₒ*)²+(a*−aₒ*)²+(b*−bₒ*)₂}

wherein L* indicates lightness and a* and b* are the chromaticity coordinates of the natural colored locks whereas L₀* indicates the lightness and a₀* and b₀* are the chromaticity of the permed colored locks. A lower value of ΔE indicates lower selectivity of the coloration and more uniform color along the hair from the tip to the roots.

The results are reported in the table below.

| | Hair type | L* | a* | b* | ΔE |
|---|---|---|---|---|---|
| A (present | BN | 25.80 | 16.24 | 0.57 | 1.84 |
| disclosure) | SA22.9 | 24.43 | 17.05 | 1.5 | |
| B | BN | 26.08 | 16.87 | −1.10 | 5.01 |
| (comparative) | SA22.9 | 21.91 | 15.81 | 1.47 | |

The results show that the selectivity (ΔE) was substantially lower for the composition of the present disclosure, indicating a more uniform coloration along the hair.

What is claimed is:

1. A dye composition for keratin fibers, comprising, in a medium suitable for dyeing:
(A) at least one nonionic derivative of cellulose comprising at least one hydrophobic substituent comprising from 8 to 30 carbon atoms;
(B) at least one oxidation dye, and
(C) at least one cationic diquaternary ammonium polymer comprising repeat units of the formula (I): 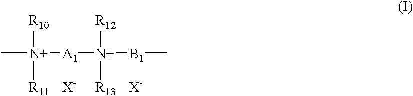
wherein:
R₁₀, R₁₁, R₁₂ and R₁₃, which may be identical or different, are chosen from linear, branched and cyclic, saturated, unsaturated and aromatic C₁-C₂₀ hydrocarbon-based radicals, linear and branched hydroxyalkyl radicals wherein the alkyl part is C₁-C₄, or alternatively, linear and branched C₁-C₆ alkyl radicals substituted with a nitrile, ester, acyl, amide, —CO—O—R₁₄-D or —CO—NH—R₁₄-D group wherein R₁₄ is an alkyl radical and D is a quaternary ammonium group, or alternatively, together or separately, form, with the nitrogen atoms to which they are attached, heterocycles optionally comprising a second heteroatom other than nitrogen;
A₁ and B₁ are chosen from linear and nonlinear, saturated and unsaturated C₂-C₂₀ radicals optionally substituted and/or interrupted with at least one entity chosen from aromatic rings, oxygen, sulphur, and groups bearing at least one of oxygen and sulphur;
X⁻ is an organic or mineral anion;
A₁, R₁₀ and R₁₂ can form, with the two nitrogen atoms to which they are attached, a piperazine ring;
wherein, if A₁ is chosen from linear and branched, saturated and unsaturated alkylene and hydroxyalkylene radicals, then B₁ can also be chosen from —(CH₂)ₙ—CO-D-OC—(CH₂)ₙ— groups wherein n ranges from 1 to 100, and D is chosen from glycols, bissecondary diamines, bisprimary diamines, and ureylene residues.

2. The dye composition according to claim 1, wherein the at least one nonionic derivative of cellulose is a hydroxyethylcellulose substituted with at least one hydrophobic substituent comprising from 8 to 30 carbon atoms.

3. The dye composition according to claim 1, wherein the at least one hydrophobic substituent is chosen from C₁₀-C₂₂ alkyl groups.

4. The dye composition according to claim 3, wherein the at least one hydrophobic substituent is chosen from cetyl groups.

5. The dye composition according to claim 1, wherein the degree of hydrophobic substitution ranges from 0.1% to 10% by weight of the total weight of the polymer.

6. The dye composition according to claim 5, wherein the degree of hydrophobic substitution ranges from 0.4% to 0.8% by weight of the total weight of the polymer.

7. The dye composition according to claim 1, wherein the at least one nonionic derivative of cellulose is present in an amount ranging from 0.01% to 10% by weight.

8. The dye composition according to claim 1, wherein the at least one nonionic derivative of cellulose is present in an amount ranging from 0.1% to 1% by weight, relative to the total weight of the composition.

9. The dye composition according to claim 1, wherein the at least one oxidation dye is chosen from oxidation bases, oxidation couplers, and addition salts thereof.

10. The dye composition according to claim 9, wherein the at least one oxidation base is chosen from para-phenylenediamines, bisphenylalkylenediamines, para-aminophenols, bis-para-aminophenols, ortho-aminophenols, heterocyclic bases, and addition salts thereof.

11. The dye composition according to claim 1, wherein the at least one oxidation coupler is chosen from benzene couplers, heterocyclic couplers, naphthalene couplers, and addition salts thereof.

12. The dye composition according to claim 11, wherein the at least one benzene coupler is chosen from meta-aminophenols, meta-phenylenediamines, meta-diphenols, and addition salts thereof.

13. The dye composition according to claim 9, wherein the at least one oxidation coupler is present in an amount ranging from 0.001% to 20% by weight.

14. The dye composition according to claim 13, wherein the at least one oxidation coupler is present in an amount ranging from 0.01% to 5% by weight, relative to the total weight of the composition.

15. The dye composition according to claim 1, wherein the at least one cationic polymer has a charge density of at least 1 meq/g.

16. The dye composition according to claim 1, wherein the at least one cationic diquaternary ammonium polymer is chosen from polymers with repeat units of formulae (W) and (U):

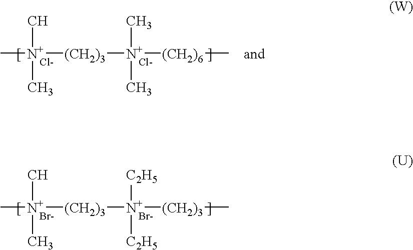

17. The dye composition according to claim 1, wherein the at least one cationic diquaternary ammonium polymer is present in an amount ranging from 0.01% to 10% by weight, relative to the weight of the composition.

18. The dye composition according to claim 17, wherein the at least one cationic polymer is present in an amount ranging from 0.05% to 6% by weight, relative to the weight of the composition.

19. The dye composition according to claim 1, further comprising at least one direct dye chosen from nitrobenzene dyes, azo direct dyes, methine direct dyes, anthraquinone dyes, xanthene dyes, triarylmethane dyes, and addition salts thereof.

20. The dye composition according to claim 1, further comprising at least one alkanolamide.

21. The dye composition according to claim 1, further comprising at least one oxidizing agent.

22. A process for the oxidation dyeing of keratin fibers, comprising
applying to the fibers, in the presence of at least one oxidizing agent, for a period of time sufficient to develop the desired color, a dye composition comprising, in a medium suitable for dyeing:
(A) at least one nonionic derivative of cellulose comprising at least one hydrophobic substituent comprising from 8 to 30 carbon atoms;
(B) at least one oxidation dye, and
(C) at least one cationic diquaternary ammonium polymer comprising repeat units of the formula (I): 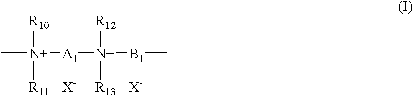
wherein:
R₁₀, R₁₁, R₁₂ and R₁₃, which may be identical or different, are chosen from linear, branched and cyclic, saturated, unsaturated and aromatic C₁-C₂₀ hydrocarbon-based radicals, linear and branched hydroxyalkyl radicals wherein the alkyl part is C₁-C₄, or alternatively, linear and branched C₁-C₆ alkyl radicals substituted with a nitrile, ester, acyl, amide, —CO—O—R₁₄-D or —CO—NH—R₁₄-D group wherein R₁₄ is an alkyl radical and D is a quaternary ammonium group, or alternatively, together or separately, form, with the nitrogen atoms to which they are attached, heterocycles optionally comprising a second heteroatom other than nitrogen;
A₁ and B₁ are chosen from linear and nonlinear, saturated and unsaturated C₂-C₂₀ radicals optionally substituted and/or interrupted with at least one entity chosen from aromatic rings, oxygen, sulphur, and groups bearing at least one of oxygen and sulphur;
X⁻ is an organic or mineral anion;
A₁, R₁₀ and R₁₂ can form, with the two nitrogen atoms to which they are attached, a piperazine ring;
wherein, if A₁ is chosen from linear and branched, saturated and unsaturated alkylene and hydroxyalkylene radicals, then B₁ can also be chosen from —(CH₂)ₙ—CO-D-OC—(CH₂)ₙ— groups wherein n ranges from 1 to 100, and D is chosen from glycols, bissecondary diamines, bisprimary diamines, and ureylene residues.

23. A multicompartment device, comprising
at least one first compartment comprising at least one dye composition for keratin fibers, comprising, in a medium suitable for dyeing:
(A) at least one nonionic derivative of cellulose comprising at least one hydrophobic substituent comprising from 8 to 30 carbon atoms;
(B) at least one oxidation dye, and
(C) at least one cationic diquaternary ammonium polymer comprising repeat units of the formula (I): 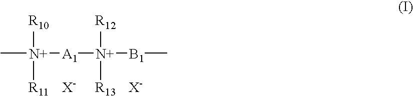
wherein:
R₁₀, R₁₁, R₁₂ and R₁₃, which may be identical or different, are chosen from linear, branched and cyclic, saturated, unsaturated and aromatic C₁-C₂₀ hydrocarbon-based radicals, linear and branched hydroxyalkyl radicals wherein the alkyl part is C₁-C₄, or alternatively, linear and branched C₁-C₆ alkyl radicals substituted with a nitrile, ester, acyl, amide, —CO—O—R₁₄-D or —CO—NH—R₁₄-D group wherein R₁₄ is an alkyl radical and D is a quaternary ammonium group, or alternatively, together or separately, form, with the nitrogen atoms to which they are attached, heterocycles optionally comprising a second heteroatom other than nitrogen;
A₁ and B₁ are chosen from linear and nonlinear, saturated and unsaturated C₂-C₂₀ radicals optionally substituted and/or interrupted with at least one entity chosen from aromatic rings, oxygen, sulphur, and groups bearing at least one of oxygen and sulphur;
X⁻ is an organic or mineral anion;
A₁, R₁₀ and R₁₂ can form, with the two nitrogen atoms to which they are attached, a piperazine ring;
wherein, if A₁ is chosen from linear and branched, saturated and unsaturated alkylene and hydroxyalkylene radicals, then B₁ can also be chosen from —(CH₂)ₙ—CO-D-OC—(CH₂)ₙ— groups wherein n ranges from 1 to 100, and D is chosen from glycols, bissecondary diamines, bisprimary diamines, and ureylene residues; and at least one second compartment comprising at least one oxidizing agent.
